(19) United States
(12) Patent Application Publication
(10) Pub. No.: US 2004/0063695 A1
(43) Pub. Date: Apr. 1, 2004

(54) Cystic fibrosis transmembrane conductance regulator protein inhibitors and uses thereof

(76) Inventors: Alan Verkman, San Francisco, CA (US); Tonghui Ma, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/262,573

(22) Filed: Sep. 30, 2002

(51) Int. Cl. A61K 31/549

(52) U.S. Cl. 514/222.5

(57) ABSTRACT

The invention provides compositions, pharmaceutical preparations and methods for inhibition of cystic fibrosis transmembrane conductance regulator protein (CFTR) that are useful for the study and treatment of CFTR-mediated diseases and conditions. The compositions and pharmaceutical preparations of the invention may comprise one or more thiazolidinone compounds, and may additionally comprise one or more pharmaceutically acceptable carriers, excipients and/or adjuvants. The methods of the invention comprise, in certain embodiments, administering to a patient suffering from a CFTR-mediated disease or condition, an efficacious amount of a thiazolidinone compound. In other embodiments the invention provides methods of inhibiting CFTR that comprise contacting cells in a subject with an effective amount of a thiazolidinone compound. In addition, the invention features a non-human animal model of CFTR-mediated disease which model is produced by administration of a thiazolidinone compound to a non-human animal in an amount sufficient to inhibit CFTR.

### BACKGROUND OF THE INVENTION

The cystic fibrosis transmembrane conductance regulator protein (CFTR) is a cAMP-activated chloride (Cl⁻) channel expressed in epithelial cells in mammalian airways, intestine, pancreas and testis. CFTR is the chloride-channel responsible for cAMP-mediated Cl− secretion. Hormones, such as a β-adrenergic agonist, or a toxin, such as cholera toxin, leads to an increase in cAMP, activation of cAMP-dependent protein kinase, and phosphorylation of the CFTR Cl⁻ channel, which causes the channel to open. An increase in cell Ca²⁺ can also activate different apical membrane channels. Phosphorylation by protein kinase C can either open or shut Cl⁻ channels in the apical membrane. CFTR is predominantly located in epithelia where it provides a pathway for the movement of Cl− ions across the apical membrane and a key point at which to regulate the rate of transepithelial salt and water transport. CFTR chloride channel function is associated with a wide spectrum of disease, including cystic fibrosis (CF) and with some forms of male infertility, polycystic kidney disease and secretory diarrhea.

The hereditary lethal disease cystic fibrosis (CF) is caused by mutations in CFTR. Observations in human cystic fibrosis (CF) patients and CF mouse models indicate the functional importance of CFTR in intestinal and pancreatic fluid transport, as well as in male fertility (Grubb et al., 1999, Physiol. Rev. 79:S193-S214; Wong, P. Y., 1997, Mol. Hum. Reprod. 4:107-110). However, the mechanisms remain unclear by which defective CFTR produces airway disease, which is the principal cause of morbidity and mortality in CF (Pilewski et al., 1999, Physiol. Rev. 79:S215-S255.). Major difficulties in understanding airway disease in CF include the inadequacy of CF mouse models, which manifest little or no airway disease, the lack of large animal models of CF, and the limited availability of human CF airways that have not been damaged by chronic infection and inflammation. High-affinity, CFTR-selective inhibitors have not been available to study airway disease mechanisms in CF or to create the CF phenotype in large animal models.

High-affinity CFTR inhibitors also have clinical applications in the therapy of secretary diarrheas and cystic kidney disease, and in inhibiting male fertility. The compounds diphenylamine-2-carboxylate (DPC) and 5-nitro-2(3-phenylpropyl-amino)benzoate (NPPB) inhibit CFTR at high concentrations but are non-specific in their inhibitory action (Cabantchik et al., 1992, Am. J Physiol. 262:C803-C827; McDonough et al., 1994, Neuron 13:623-634; Schultz et al., 1999, Physiol. Rev. 79:S109-S144.). The best CFTR inhibitor available for electrophysiological and other cell-based studies, glibenclamide, is used at concentrations of >100 μM (Sheppard et al., 1992, J. Gen. Physiol. 100:573-591; Hongre et al, 1994, Pfugers Arch. 426:284-287). However, at this concentration glibenclamide also inhibits other Cl⁻ transporters as well as K⁺ channels (Edwards et al., 1993, Br. J. Pharmacol. 110:1280-1281; Rabe et al., 1995, Pflugers Arch. 429:659-662; Yamazaki et al., 1997, Circ. Res. 81:101-109). Effective small molecule inhibitors of other ion transport proteins are known, but no small molecules with specific CFTR inhibitory ability suitable for therapy of secretory diseases have been available.

There is accordingly a need in for CTFR inhibitor compounds and methods of using such compounds for development of animal models useful in the study and treatment of CF and the treatment and control of secretory disorders. The present invention addresses these needs, as well as others, and overcomes deficiencies found in the background art.

### SUMMARY OF THE INVENTION

The invention provides compositions, pharmaceutical preparations and methods for inhibition of cystic fibrosis transmembrane conductance regulator protein (CFTR) that are useful for the study and treatment of CFTR-mediated diseases and conditions. The compositions and pharmaceutical preparations of the invention may comprise one or more thiazolidinone compounds, and may additionally comprise one or more pharmaceutically acceptable carriers, excipients and/or adjuvants. The methods of the invention comprise, in certain embodiments, administering to a patient suffering from a CFTR-mediated disease or condition, an efficacious amount of a thiazolidinone compound. In other embodiments the invention provides methods of inhibiting CFTR that comprise contacting cells in a subject with an effective amount of a thiazolidinone compound. In addition, the invention features a non-human animal model of CFTR-mediated disease which model is produced by administration of a thiazolidinone compound to a non-human animal in an amount sufficient to inhibit CFTR.

These and other objects and advantages of the invention will be apparent from the detailed description below.

### BRIEF DESCRIPTION OF THE DRAWINGS

The invention will be more fully understood by reference to the following drawings, which are for illustrative purposes only.

FIG. 1A is a schematic representation of a screening technique used for detection of CFTR inhibitors. CFTR was maximally stimulated by multiple agonists in stably transfected epithelial cells co-expressing human CFTR and a yellow fluorescent protein (YFP) having Cl⁻/I⁻ sensitive fluorescence. After addition of a test compound, I⁻ influx was induced by adding an I⁻ containing solution.

FIG. 1B is a graphical illustration of representative fluorescence data from individual wells using the screening technique of FIG. 1A, showing controls (no activator, no test compound), inactive compounds and active CFTR inhibitor compounds.

FIG. 1C shows chemical structures of 2-thioxo-4-thiazolidinone CFTR inhibitors identified by the screening technique of FIG. 1A.

FIG. 1D shows chemical structures of Ring 2 of the 2-thioxo-4-thiazolidinone analogs having the greatest CFTR inhibitory activity. The complete 2-thioxo-4-thiazolidinone structure is shown in FIG. 1C. Relative potencies were: 0.2 (CFTRᵢₙₕ-020), 0.3 (CFTRᵢₙₕ-029), 1.0 (CFTRᵢₙₕ-172), 0.2 (CFTRᵢₙₕ-185), 0.1 (CFTRᵢₙₕ-214) and 0.1 (CFTRᵢₙₕ-236).

FIG. 2A is a graphical representation of relative fluorescence versus time using the screening technique of FIG. 1A for the CFTR inhibitor 3-[(3-trifluoromethyl)phenyl]-5-[(4-carboxyphenyl)methylene]-2-thioxo-4-thiazolidinone (referred to herein as CFTRᵢₙₕ-172) at several concentrations.

FIG. 2B is a graphical representation of the time course of inhibition showing CFTR-mediated I- transport rates at different times after addition of 2 μM CFTRᵢₙₕ-172. The inset is a graphical representation of the time course of inhibition reversal showing I⁻ transport rates at different times after washout of 1 μM CFTRᵢₙₕ-172. Mean ± SE from three sets of experiments.

FIG. 2C is a graphical representation of inhibition of CFTR after stimulation by different agonists, including benzoflavone and benzimidazolone UCCF compounds (UC_{CF}-029 (2-(4-pyridinium)benzo[h]4H-chromen-4-one bisulfate) and UC_{CF}-853 (Galietta et al. 2001 J. Biol. Chem. 276:19723-19728), genistein, CPT-cAMP, 8-methoxypsoralen (8-MPO), 8-cyclopentyl-1,3-dipropylxanthine (CPX) (all 50 μM) (±SE from three sets of experiments). Filled bars show agonist, and open bars show agonist with 5 μM CFTRᵢₙₕ-172.

FIG. 3A is a graphical representation of CFTRᵢₙₕ-172 inhibition of short-circuit current in permeabilized FRT cells expressing human CFTR. CFTR was stimulated by 100 μM CPT-cAMP.

FIG. 3B graphically provides a summary of dose-inhibition data for CFTRᵢₙₕ-172 (circles) and glibenclamide (squares) (SE, three sets of experiments).

FIG. 3C graphically illustrates CFTRᵢₙₕ-172 inhibition of short-circuit current in primary culture of (non-permeabilized) human bronchial epithelial cells. Inhibitor was added in apical bathing solution (left panel) or basolateral and then apical solutions (right panel).

FIG. 3D is a graphical representation of whole-cell patch clamp of CFTR-expressing FRT cells showing membrane currents elicited at +80 mV (open circles) and −100 mV (closed circles). CFTR was stimulated by 5 μM forskolin followed by addition of 2 μM CFTRᵢₙₕ-172.

FIG. 3E is a graphic illustration showing that alternate stimulation was interrupted (a-c) to apply graded membrane potentials.

FIG. 3F is a graphical representation of current-voltage relationships under basal conditions (control, open circles), after forskolin stimulation (filled circles), and following addition of 0.2 μM CFTRᵢₙₕ-172 giving ˜50% inhibition (open triangles).

FIG. 4A is a graphical representation of UTP- (100 μM) stimulated Ca²⁺-dependent Cl⁻ secretion measured in short-circuit current measurements on airway epithelial cells in the absence and presence of 5 μM of CFTRᵢₙₕ-172.

FIG. 4B is a graphical representation of volume-activated Cl⁻ current (hypotonic 250 mosM/kg H₂O) measured in whole-cell patch clamp experiments on FRT cells. Currents were recorded in the absence and presence of 5 μM CFTRᵢₙₕ-172.

FIG. 4C is a graphical representation of ³H-vincristine accumulation in 9HTEo-/Dx cells with upregulated MDR-1expression. Intracellular vincristine was measured with and without verapamil (100 μM) or CFTRᵢₙₕ-172 (5 μM) (SE, n=3).

FIG. 4D is a graphical illustration showing a representative membrane potential recording from a pancreatic β cell (INS-1) perfused extracellularly with CFTRᵢₙₕ-172, diazoxide (100 μM), and glibenclamide (10 μM).

FIG. 4E is a graphical representation of averaged changes in membrane potential (ΔmV) caused by maneuvers indicated in FIG. 4D (SE, n=4). FIG. 5A is a photograph of isolated mouse ileal loops at six hours after lumenal injection of 1 μg cholera toxin without (top) and with (middle) intraperitoneal injection of CFTRᵢₙₕ-172 (150 μg/kg). A saline control (no cholera toxin, bottom) is shown for comparison.

FIG. 5A is a photograph of isolated mouse ileal loops at six hours after lumenal injection of 1 μg cholera toxin without (top) and with (middle) intraperitoneal injection of CFTRᵢₙₕ-172 (150 μg/kg). A saline control (no cholera toxin, bottom) is shown for comparison.

FIG. 5B graphically illustrates ileal loop weight at six hours, with a mean ± SE (n=6-8 mice) with 14-16 loops studied. For the inactive analog, the 4-carboxyphenyl group in CFTRᵢₙₕ-172 was replaced by 3-methoxy-4-methoxyvinylphenyl (SE, 6-8 mice per group, * p<0.001, ANOVA).

FIG. 5C graphically illustrates the ratio of weight of entire small intestine at six hours after oral gavage before vs. after luminal fluid removal (SE, 4 mice per group, p<0.001).

FIG. 5D is a graphical illustration showing a representative CFTRᵢₙₕ-172 inhibition short-circuit current after amiloride addition and stimulation by forskolin (20 μM) in isolated rat colonic mucosa. CFTRᵢₙₕ-172 added to serosal and then mucosal surfaces as indicated (n=4).

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It should be noted that, as used herein and in the appended claims, the singular forms “a”, “and”, and “the” include plural referents unless the context clearly dictates otherwise. Thus, for example, reference to “an inhibitor” includes a plurality of such inhibitors, and reference to “the cell” includes reference to one or more cells and equivalents thereof known to those skilled in the art, and so forth.

The publications discussed herein are provided solely for their disclosure prior to the filing date of the present application, and are incorporated herein by reference. Nothing herein is to be construed as an admission that the present invention is not entitled to antedate such publication by virtue of prior invention. Further, the dates of publication provided may be different from the actual publication dates that may need to be independently confirmed.

The definitions used herein are provided for reason of clarity, and should not be considered as limiting. The technical and scientific terms used herein are intended to have the same meaning as commonly understood by those of ordinary skill in the art to which the invention pertains.

### DETAILED DESCRIPTION OF THE INVENTION

The invention is based on the discovery of thiazolidinone compounds that are high-affinity CFTR inhibitors. The structure of the compounds of the invention, as well as pharmaceutical formulations and methods of use are described in more detail below.

Definitions

A “cystic fibrosis transmembrane conductance regulator protein-mediated condition or symptom” or “CFTR-mediated condition or symptom” means any condition, disorder or disease, or symptom of such condition, disorder, or disease, that results from activity of cystic fibrosis transmembrane conductance regulator protein (CFTR), e.g., activity of CFTR in ion transport. Such conditions, disorders, diseases, or symptoms thereof are treatable by inhibition of CFTR activity, e.g., inhibition of CFTR ion transport. CFTR activity has been implicated in, for example, intestinal secretion in response to various agonists, including cholera toxin (see, e.g., Snyder et al. 1982 Bull. World Health Organ. 60:605-613; Chao et al. 1994 EMBO J. 13:1065-1072; Kimberg et al. 1971 J. Clin. Invest.50:1218-1230).

A “CFTR inhibitor” as used herein is a compound that reduces the efficiency of ion transport by CFTR, particularly with respect to transport of chloride ions by CFTR. Preferably CFTR inhibitors of the invention are specific CFTR inhibitors, i.e., compounds that inhibit CFTR activity without significantly or adversely affecting activity of other ion transporters, e.g., other chloride transporters, potassium transporters, and the like. Preferably the CFTR inhibitors are high-affinity CFTR inhibitors, e.g., have an affinity for CFTR of at least about one micromolar, usually about one to five micromolar.

“Treating” or “treatment” of a condition or disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

The terms “subject” and “patient” mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

“Mammal” means a member or members of any mammalian species, and includes, by way of example, canines; felines; equines; bovines; ovines; rodentia, etc. and primates, particularly humans. Non-human animal models, particularly mammals, e.g. primat, murine, lagomorpha, etc. may be used for experimental investigations.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular terpene or terpenoid compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4methylbicyclo>2.2.2!oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Pro-drugs” means any compound that releases an active parent drug according to formula (I) in vivo when such prodrug is administered to a mammalian subject. Prodrugs of a compound of formula (I) are prepared by modifying functional groups present in the compound of formula (I) in such a way that the modifications may be cleaved in vivo to release the parent compound. Prodrugs include compounds of formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups in compounds of formula (I), and the like.

The term “organic group” and “organic radical” as used herein means any carbon-containing group, including hydrocarbon groups that are classified as an aliphatic group, cyclic group, aromatic group, functionalized derivatives thereof and/or various combination thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈ ) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Preferred halogens are chloro and fluoro.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C1 to C7 alkyl, C1 to C7 alkoxy, C1 to C7 acyl, C1 to C7 acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C1 to C6 alkyl)carboxamide, protected N-(C1 to C6 alkyl)carboxamide, N,N-di(C1 to C6 alkyl)carboxamide, trifluoromethyl, N-((C1 to C6 alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to five and six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms. These five-membered or six-membered rings may be fully unsaturated.

Furthermore, the above optionally substituted five-membered or six-membered rings can optionally be fused to a aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C1 to C4 alkoxy group, similarly, “lower alkylthio” means a C1 to C4 alkylthio group.

The term “(monosubstituted)amino” refers to an amino group with one substituent chosen from the group consisting of phenyl, substituted phenyl, alkyl, substituted alkyl, C1 to C4 acyl, C2 to C7 alkenyl, C2 to C7 substituted alkenyl, C2 to C7 alkynyl, C7 to C16 alkylaryl, C7 to C16 substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C1 to C7 acyl, C2 to C7 alkenyl, C2 to C7 alkynyl, C7 to C16 alkylaryl, C7 to C16 substituted alkylaryl and heteroaryl. The two substituents can be the same or different.

The term “heteroaryl(alkyl)” denotes an alkyl group as defined above, substituted at any position by a heteroaryl group, as above defined.

“Optional” or “optionally” means that the subsequently described event, circumstance, feature or element may, but need not, occur, and that the description includes instances where the event or circumstance occurs and instances in which it does not. For example, “heterocyclo group optionally mono- or di- substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

Overview

The invention provides thiazolidinone compositions and methods of their use in high affinity inhibition of cystic fibrosis transmembrane conductance regulator protein (CFTR) and for the study and treatment of CFTR-mediated diseases and conditions. The discovery of the subject thiazolidinone compounds was based on screening of numerous potential candidate compounds using an assay designed to identify CFTR inhibitors that interact directly with CFTR. Without being held to any particular theory or mode of operation, since multiple CFTR activators that work on different activating pathways were included in the studies leading to identification of the subject compounds, the inhibitory compounds of the invention likely effect inhibition by acting at or near the CFTR Cl⁻ transporting pathway. A screening of 50,000 diverse compounds identified several 2-thioxo-4-thiazolidinone compounds as effective CFTR inhibitors. These compounds are unrelated chemically and structurally to previously known CFTR activators or to the previously known CFTR inhibitors DPC, NPPB or glibenclamide. The most potent CFTR inhibitor identified from screening had a K₁ of ˜300 nM for inhibition of Cl⁻ current in human airway cells. Inhibition was rapid, reversible and CFTR-specific.

The compositions and methods of the invention will now be described in more detail.

Thiazolidinone Compounds

The thiazolidinone compounds used in the compositions and methods of the invention comprise a heterocyclic ring of five or more atoms, including an aryl substituted nitrogen, at least one sulfur, oxygen or selenium heteroatom, and one or more carbonyl or thiocarbonyl groups associated with the heterocyclic ring. More specifically, the subject thiazolidinone compounds may comprise the formula

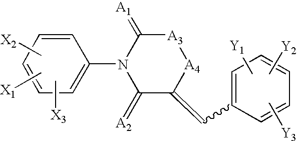

wherein X₁, X₂ and X₃ each individually are hydrogen, any organic group, any halo group, or a nitro group, azo group, hydroxyl group or thio group, Y₁, Y₂ and Y₃ each individually comprise hydrogen, any organic group, any halo group, or a nitro group, azo group, hydroxyl group or thio group, A₁ and A₂ each individually are oxygen or sulfur, A₃ is sulfur or selenium, and A₄ comprises one or more carbons or heteroatoms and may be present or absent. Where A₄ is absent the central heterocyclic ring is a five membered ring.

In certain embodiments, the thiazolidinone compounds comprise the formula:

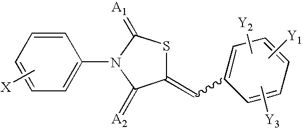

wherein X is hydrogen, any organic group, any halo group, or a nitro group, azo group, hydroxyl group or thio group, Y₁, Y₂ and Y₃ each individually are hydrogen, any organic group, any halo group, or a nitro group, azo group, hydroxyl group or thio group, and A₁ and A₂ each independently are oxygen or sulfur. In specific embodiments, X may be an electron withdrawing group, and may comprise a haloalkyl group, dihaloalkyl group, trihaloalkyl group (e.g., trifluoroalkyl group) or a fluoro group. Y₁ may be selected from the group consisting of alkyl, hydroxyl, carboxyl, nitro, carbonate, carbamate, alkoxy, alkylcarbonyl, and halo groups, Y₂ may be selected from the group consisting of hydroxyl and bromo groups, and Y₃ may be selected from the group consisting of hydrogen and a nitro group.

The subject thiazolidinone compounds in many embodiments may comprise 3-aryl-5-arylmethylene-2-thioxo-4-thiazolidinones of the formula

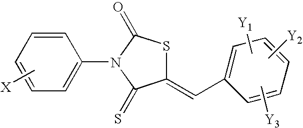

wherein X is any electronegative or electron withdrawing group, and Y₁, Y₂ and Y₃ each individually are hydrogen, alkyl, hydroxyl, carboxyl, nitro, carbonate, carbamate, alkoxy, alkylcarbonyl, or halo groups. In one embodiment X is at position selected from 2, 3, or 4; Y₁, is at a position selected from 2, 3, or 4; and Y₂ and Y₃ may be hydrogen. The 3-aryl-5-arylmethylene-2-thioxo-4-thiazolidinones may more specifically have the formula

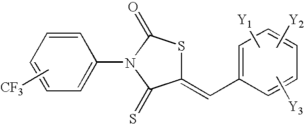

wherein Y₁-Y₃ are as described above. In one embodiment trifluromethyl group is at a position selected from 2, 3, or 4; Y₁ is at a position selected from 2, 3, or 4; where Y₂ and Y₃ may be hydrogen in this embodiment.

In some embodiments of the invention, the thiazolidinone compounds may comprise:

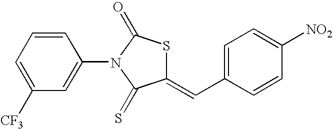

3-[(3-trifluoromethyl)phenyl]-5-[(4-nitrophenyl)methylene]-2-thioxo-4-thiazolidinone;

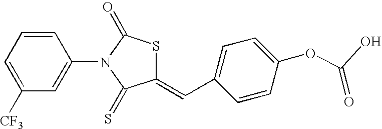

3-[(3-trifluoromethyl)phenyl]-5-[(4-oxycarboxyphenyl)methylene]-2-thioxo-4-thiazolidinone;

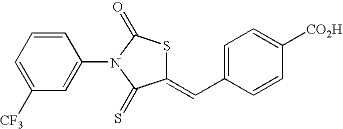

3-[(3-trifluoromethyl)phenyl]-5-[(4-carboxyphenyl)methylene]-2-thioxo-4-thiazolidinone;

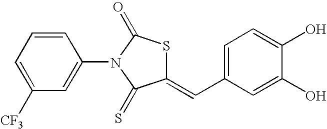

3-[(3-trifluoromethyl)phenyl]-5-[(3,4-dihydroxyphenyl)methylene]-2-thioxo-4-thiazolidinone;

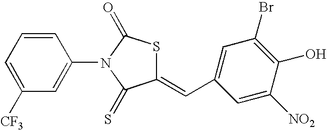

3-[(3-trifluoromethyl)phenyl]-5-[(3,5-dibromo-4-hydroxyphenyl)methylenel-2-thioxo-4-thiazolidinone; and

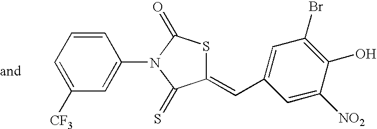

3-[(3-trifluoromethyl)phenyl]-5-[(3-bromo-4-hydroxy-5-nitrophenyl)methylene]-2-thioxo-4-thiazolidinone. The trifluromethyl group may be at positions 2, 3, or 4 in any of the above-recited compounds.

Pharmaceutical Preparations

Also provided by the invention are pharmaceutical preparations of the subject thiazolidinone compounds described above. The subject compounds can be incorporated into a variety of formulations for therapeutic administration by a variety of routes. More particularly, the compounds of the present invention can be formulated into pharmaceutical compositions by combination with appropriate, pharmaceutically acceptable carriers, diluents, excipients and/or adjuvants, and may be formulated into preparations in solid, semi-solid, liquid or gaseous forms, such as tablets, capsules, powders, granules, ointments, solutions, suppositories, injections, inhalants and aerosols. Preferably, the formulations are free of detectable DMSO (dimethyl sulfoxide), which is not a pharmaceutically acceptable carrier, diluent, excipient, or adjuvant. The formulations may be designed for administration to subjects or patients in need thereof via a number of different routes, including oral, buccal, rectal, parenteral, intraperitoneal, intradermal, transdermal, intracheal, etc., administration.

In pharmaceutical dosage forms, the subject compounds of the invention may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the subject compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The subject compounds of the invention can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds of the invention can be utilized in aerosol formulation to be administered via inhalation. The compounds of the present invention can be formulated into pressurized acceptable propellants such as dichlorodifluoromethane, propane, nitrogen and the like.

Furthermore, the subject compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

Depending on the subject and condition being treated and on the administration route, the subject compounds may be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. The inventors have found that cholera toxin-induced intestinal fluid secretion in mice is effectively blocked by a single intraperitoneal dose of about 10-20 micrograms with a dosage of about ten times greater being effective in rats. Thus, for example, oral dosages may be about ten times the injection dose. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

For use in the subject methods, the subject compounds may be formulated with other pharmaceutically active agents, including other CFTR-inhibiting agents.

Pharmaceutically acceptable excipients usable with the invention, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Kits with unit doses of the subject compounds, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

Conditions Amenable to Treatment Using the CFTR Inhibitors of the Invention

The CFTR inhibitors disclosed herein are useful in the treatment of a CFTR-mediated condition, i.e., any condition, disorder or disease, or symptom of such condition, disorder, or disease, that results from activity of CFTR, e.g., activity of CFTR in ion transport. Such conditions, disorders, diseases, or symptoms thereof are amenable to treatment by inhibition of CFTR activity, e.g., inhibition of CFTR ion transport.

In one embodiment, the CFTR inhibitors of the invention are used in the treatment of conditions associated with aberrantly increased intestinal secretion, particularly acute aberrantly increased intestinal secretion. CFTR activity has been implicated in intestinal secretion in response to various agonists, including cholera toxin (see, e.g., Snyder et al. 1982 Bull. World Health Organ. 60:605-613; Chao et al. 1994 EMBO J. 13:1065-1072; Kimberg et al. 1971 J Clin. Invest.50:1218-1230). Thus CFTR inhibitors of the invention can be administered in an amount effective to inhibit CFTR ion transport and thus decrease intestinal fluid secretion.

Thus, CFTR inhibitors can be used in the treatment of intestinal inflammatory disorders and diarrhea, particularly secretory diarrhea. Secretory diarrhea is the biggest cause of infant death in developing countries, with about 5 million deaths annually (Gabriel et al., 1994 Science 266: 107-109). Several studies, including those using CF mice, indicate that CFTR is the final common pathway for intestinal chloride ion (and thus fluid) secretion in response to various agonists (Snyder et al., 1982, Bull. World Health Organ. 60: 605-613; Chao et al., 1994 EMBO. J. 13: 1065-1072; and Kimberg et al., 1971, J. Clin. Invest. 50: 1218-1230.). The mouse models of intestinal fluid secretion used herein indicate that CFTR inhibition by systemic administration of the inhibitor at a non-toxic dose effectively blocked intestinal fluid secretion induced by cholera toxin (see Examples).

Diarrhea can result from exposure to a variety of pathogens or agents including, without limitation, cholera toxin (Vibrio cholera), enterotoxigenic E. coli (ETEC), food poisoning, or other toxin exposure that results in increased intestinal secretion mediated by CFTR.

CFTR inhibitors may also be useful in the treatment of diarrhea associated with AIDS (e.g., AIDS-related diarrhea), and inflammatory gastrointestinal disorders, such as ulcerative colitis, inflammatory bowel disease (IBD), Crohn's disease, and the like. It has been reported that intestinal inflammation modulates the expression of three major mediators of intestinal salt transport and may contribute to diarrhea in ulcerative colitis both by increasing transepithelial Cl− secretion and by inhibiting the epithelial NaCl absorption (see, e.g., Lohi et al. Am J Physiol Gastrointest Liver Physiol 2002 Sep;283(3):G567-75).

CFTR inhibitors of the invention can also be used in treatment of conditions such as polycystic kidney disease, and find further use as male infertility drugs, by inhibition of CFTR activity in the testis.

CFTR inhibitors of the invention can be further screened in larger animal models (e.g., the rabbit model described in Spira et al., 1981, Infect. Immun. 32:739-747.). In addition, analysis of stool output using live Vibrio cholerae can also be examined to further characterize the CFTR inhibitors of the invention.

Non-Human Animal Models and Human Tissue Models of CFTR-Deficiencies

The CFTR inhibitors of the invention can also be used to generate non-human animal models of disease, where the disease is associated with decreased CFTR function (e.g., decreased ion transport). There is increasing evidence that defective fluid and macromolecular secretion by airway submucosal glands leads to impaired mucociliary and bacterial clearance in CFTR-deficient subjects, particularly in those affected with cystic fibrosis (CF); however, functional studies in human airway glands have been restricted to severely diseased airways obtained at the time of lung transplantation (Jayaraman et al. 2001 Proc. Natl. Acad. Sci. USA 98:8119-8123). Acute CFTR inhibition permits determination of the role of CFTR in water, salt and macromolecule secretion by submucosal glands. High-affinity CFTR inhibitors permit the pharmacological creation of non-human animal models that mimic CFTR-deficiency in humans, e.g., mimics the human CF phenotype. In particular, large animal models of CFTR deficiency (e.g., CF) find particular use in elucidating the pathophysiology of initiation and progression of airway disease in CF, and in evaluating the efficacy of CF therapies, e.g., screening candidate agents for treatment of CFTR-deficiencies or symptoms thereof.

Inhibition of CFTR ion transport can be manifested in airway and pancreatic disorders, as well as infertility in males. For example, inhibition of CFTR channels in the lungs and airways influences airway surface fluids leading to accumulation of mucus, which in turn plugs airways and collects heavily on the lung walls, providing a prime environment for infection to occur, which in turn can lead to chronic lung disease. This same phenomenon occurs in the pancreas, where the accumulated mucus disrupts the exocrine function of the pancreas and prevents essential food-processing enzymes from reaching the intestines.

Such non-human animal models can be generated by administration of an amount of a CFTR inhibitor effective to decrease CFTR activity in ion transport. Of particular interest is the use of the CFTR inhibitors of the invention to induce the cystic fibrosis (CF) phenotype in a non-human animal. Administration of an amount of a CFTR inhibitor effective to inhibit CFTR receptors in, for example, lung effectively mimics the CFTR defect found in CF. Routes of delivery for CFTR inhibitor are discussed in detail above. Depending on the non-human animal used, the subject compounds may be administered in dosages of, for example, 50 to 500 μg/kg body weight one to three times a day by an intraperitoneal, subcutaneous, or other route to generate the non-human animal models. Oral dosages may be up to about ten times the intraperitoneal or subcutaneous dose.

Non-human animal models of CFTR-associated disease can be used as models of any appropriate condition associated with decreased CFTR activity. Such conditions include those that are associated with CFTR mutations, which mutations result in abnormalities in epithelial ion and water transport. These abnormalities can in turn be associated with derangements in airway mucociliary clearance, as well as in other mucosal epithelia and ductal epithelia. Conditions that can be pharmacologically modeled by inducing a CFTR-deficient phenotype in a non-human animal include, without limitation, cystic fibrosis (including atypical CF), idiopathic chronic pancreatitis, vas deferens defects, mild pulmonary disease, asthma, and the like. For a review of disorders associated with impaired CFTR function, see, e.g., Noone et al. Respir Res 2 328-332 (2001). CFTR inhibitor-generated non-human animal models can also serve as models of microbial infection (e.g., bacterial, viral, or fungal infection, particularly respiratory infections) in a CFTR-deficient subject. In one embodiment of particular interest, the CFTR inhibitors of the invention are used to pharmacologically induce the cystic fibrosis (CF) phenotype.

Animals suitable for use in the production of the animal models of the invention include any animal, particularly a mammal, e.g., non-human primates (e.g., monkey, chimpanzee, gorilla, and the like), rodents (e.g., rats, mice, gerbils, hamsters, ferrets, and the like), lagomorphs, swine (e.g., pig, miniature pig), equine, canine, feline, and the like. Large animals are of particular interest.

The CFTR inhibitors can also be contacted with isolated human tissue to create ex vivo models of disease. Such tissue is contacted with an amount of a CFTR inhibitor effective to decrease CFTR activity in the tissue, which may be for as little as 15 minutes, or as much as two hours, or more Human tissues of interest include, without limitation, lung (including trachea and airways), liver, pancreas, testis, and the like. Physiological, biochemical, genomic or other studies can be carried out on the inhibitor-treated tissue to identify novel therapeutic target molecules that are important in the pathophysiology of a disease. For example, isolated tissue from humans without CF can be exposed to inhibitor sufficient to induce the CF phenotype and such studies can be carried out to identify novel therapeutic target molecules that are important in the pathophysiology of CF.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following methods and materials are used in the examples below.

Cell Lines, Mice and Compounds

Fischer rat thyroid (FRT) cells coexpressing human wildtype CFTR and the halide indicator YFP-H148Q were generated as described previously (Galietta et al. 2001 J. Biol. Chem. 276:19723-19728). Cells were plated in 96-well black-walled microplates (Corning Costar) at a density of 20,000 cells per well in Coon's modified F12 medium supplemented with 5% fetal calf serum, 2 mM L-glutamine, 100 U/ml penicillin, and 100 μg/ml streptomycin. Assays were done at 48 h after plating at which time cells were just confluent (˜40,000 cells per well).

Initial screening was done using a diverse collection of 50,000 drug-like compounds from ChemBridge (San Diego, Calif.) obtained as 10 mM stock solutions in DMSO and diluted to 100 mM in 96-well microplates. Structure-activity analysis was done on analogs purchased from ChemBridge and ChemDiv (San Diego, Calif.).

Wildtype and cystic fibrosis (ΔF508 homozygous mutant) mice were bred by the CF Animal Core facility at U.C.S.F. Animal protocols were approved by the U.C.S.F. Committee on Animal Research.

Synthesis of 2-thioxo-4-thiazolidinone Analogs

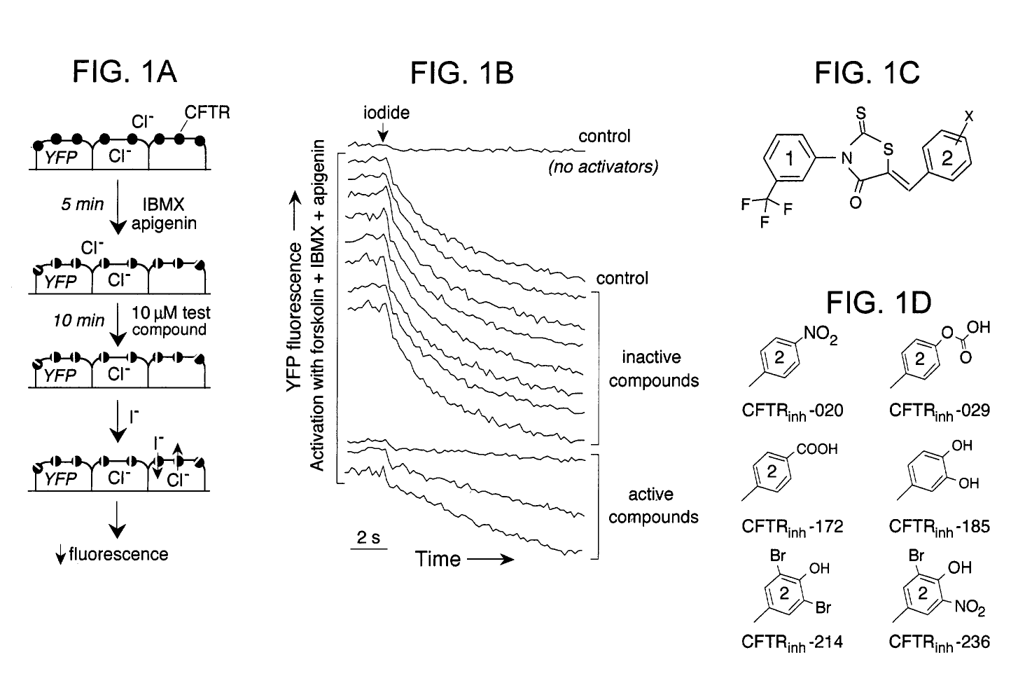

Synthesis of 3-[(3-trifluoromethyl)phenyl]-5-[(4-carboxyphenyl)methylene]-2-thioxo-4-thiazolidinone (referred to herein as CFTRᵢₙₕ-172) (see FIG. 1C) and analogs with different positions of the trifluoromethyl and carboxy substituents (see, e.g., FIG. 1D) was accomplished by Knoevenagel condensation of 2-thioxo-3-[a-trifluoromethyl-4-phenyl]-4-thiazolidinone (a=2, 3 or 4) with b-carboxybenzaldehyde (b=2, 3 or 4) in the presence of piperidine. Precipitate were filtered, washed with ethanol, dried and recrystallized 2-3 times from ethanol to give bright yellow crystals (70-85% yields). Structures were confirmed by ¹H-NMR. Purity was >99% as judged by thin layer chromatography and HPLC.

Screening Procedures

Assays were done using a customized screening system (Beckman) consisting of a 3-meter robotic arm, CO₂ incubator, plate washer, liquid handling workstation, bar code reader, delidding station, and two FluoStar fluorescence plate readers (BMG Labtechnologies, Offenburg, Germany), each equipped with two syringe pumps and HQ500/20X (500±10 nm) excitation and HQ535/30M (535±15 nm) emission filters (Chroma). The robotic system was integrated using SAMI version 3.3 software (Beckman) modified for two plate readers. Custom software was written in VBA (Visual Basic for Applications) to compute baseline-subtracted, normalized fluorescence slopes (giving halide influx rates) from stored data files.

The assay was set-up by loading the incubator (37° C., 90% humidity, 5% CO₂) with 40-60 96-well plates containing the FRT cells, and loading a carousel with 96-well plates containing test compounds and disposable plastic pipette tips. To initiate the assay, each well of a 96-well plate was washed 3 times in PBS (300 μl/wash), leaving 50 μl PBS. Ten μl of a CFTR-activating cocktail (5 μM forskolin, 100 μM IBMX, 25 μM apigenin in PBS) was added, and after 5 min one test compound (0.5 μl of 1 mM DMSO solution) was added to each well to give 10 μM final concentration. After 10 min, 96-well plates were transferred to a plate reader for fluorescence assay. Each well was assayed individually for CFTR-mediated I⁻ transport by recording fluorescence continuously (200 ms per point) for 2 s (baseline) and then for 12 s after rapid (<0.5 s) addition of 160 μL of isosmolar PBS in which 137 mM Cl⁻ was replaced by I⁻.

Assays of Intracellular [CAMP] and Toxicity

[cAMP] and phosphatase assays were done as reported previously (Galietta et al. 2001 J. Biol. Chem. 276:19723-19728). Cell toxicity was done by the dihydrorhodamine method at 24 hours after cell incubation with 0-1000 μM inhibitor. Animal toxicity was assessed by measurement of serum chemistries and hematology (U.C.S.F. Clinical Laboratory) in mice at 5 days after daily intraperitoneal injections with 0-100 μg/kg inhibitor.

MDR-1 Activity

MDR-1 activity was evaluated by measuring ³H-vincristine accumulation in an immortalized human tracheal cell line, 9HTEo-/Dx, in which the endogenous expression of MDR-1 was upregulated by selection in increasing concentrations of doxorubicin (Rasola et al. 1994 J. Biol. Chem. 269:1432-1436). Cells were seeded in 24-well microplates (200,000 cells/well). After 48 hours, cells were washed with a solution containing (in mM): 130 NaCl, 2 KCl, 1 KH₂PO₄, 2 CaCl₂, 2 MgCl₂, 10 Na-Hepes (pH 7.3) and 10 glucose, and incubated for 1 hour at 37° C. with 200 μl of the same solution containing ³H-vincristine (0.7 μM; 1 μCi/ml). Cells were then washed three times with ice-cold solution and lysed in 0.25 M NaOH. Vincristine content was determined by scintillation counting.

Short-Circuit Current Tests

Snapwell inserts containing CFTR-expressing FRT cells or human bronchial epithelial cells were mounted in an Ussing chamber system. For FRT cells the hemichambers were filled with 5 ml of 75 mM NaCl and 75 mM Na gluconate (apical) and 150 mM NaCl (basolateral) (pH 7.3), and the basolateral membrane was permeabilized with 250 μg/ml amphotericin B (Galietta et al. 2001 J Biol. Chem. 276:19723-19728). For bronchial epithelial cells and T84 cells, both hemichambers contained a Krebs bicarbonate solution. Hemichambers were continuously bubbled with air (FRT cells) or 5% CO₂ in air (bronchial and T84 cells) and maintained at 37° C. Short-circuit current was recorded continuously using a DVC-1000 voltage clamp (World Precision Instruments, Sarasota, Fla.) using Ag/AgCl electrodes and 1 M KCl agar bridges.

Patch-Clamp Analysis of Cl⁻ Channel Activity

Membrane current was measured in a whole-cell configuration. For recordings of Cl⁻ channels, the extracellular (bath) solution contained (in mM): 150 NaCl, 1 CaCl₂, 1 MgCl₂, 10 glucose, 10 mannitol, 10 TES (pH 7.4), and the intracellular (pipette) solution contained: 120 CsCl, 1 MgCl₂, 10 TEA-Cl, 0.5 EGTA, 1 Mg-ATP, 10 Hepes (pH 7.3). CFTR was activated by forskolin (5 μM) in the extracellular solution. The time-course of membrane conductance was monitored in response to alternating voltage pulses of −100 and +80 mV. At defined times the protocol was interrupted to generate current-voltage relationships (voltage pulses from −100 to +100 mV in 20 mV increments). Volume-sensitive Cl⁻ channels were activated by a hypotonic solution (extracellular NaCl decreased to 120 NaCl; 250 mosM/kg). Calcium-sensitive Cl⁻ channels were activated in human bronchial epithelial cells by addition of 100 μM UTP to the extracellular solution.

Patch-Clamp Analysis of ATP-Sensitive K⁺ Channels

Membrane potential was recorded in the pancreatic, cell line INS-1 in which the extracellular (bath) solution contained (in mM): 130 NaCl, 2 KCl, 1 KH₂PO₄, 2 CaCl₂, 2 MgCl₂, 10 Na-Hepes (pH 7.3) and 10 glucose. The pipette contained (in mM): 140 KCl, 1 CaCl₂, 2 mM MgCl₂, 10 EGTA, 0.5 MgATP, 10 K-Hepes (pH 7.3). After achieving the whole-cell configuration, the amplifier was switched to current-clamp mode.

Intestinal Fluid Secretion and Short-circuit Current

In the first of 3 assays, fluid accumulation in ileal loops was measured (Oi et al. 2002 Proc. Natl. Acad. Sci. USA 99:3042-3046; Gorbach et al. 1971 J. Clin. Invest. 50:881-889). Mice (age 8-10 weeks, body weight 25-35 g) in a CD1 genetic background (or ΔF508 homozygous mice) were starved for 24 hrs and anaesthetized with intraperitoneal ketamine (40 mg/kg) and xylazine (8 mg/kg). Body temperature was maintained during surgery at 36-38° C. using a heating pad. A small abdominal incision was made to expose the small intestine and closed ileal loops (length 20-30 mm) proximal to the cecum were isolated by sutures. Loops were injected with 100 μl of PBS alone or PBS containing cholera toxin (1 μg). In some experiments the inhibitor (150 μg/kg) was administered by intraperitoneal injection. The abdominal incision was closed with suture and mice were allowed to recover from anesthesia. At 6 hours the mice were anesthestized, intestinal loops were exteriorized, and loop length and weight were measured after removal of mesentery and connective tissue.

In the sealed adult mouse model of secretory diarrhea mice were gavaged with cholera toxin (10 μg) in 0.1 ml of 7% bicarbonate buffer (or buffer alone) using a orogastric feeding needle (Richardson et al. 1986 Infect. Immun. 54:522-528; Gabriel et al. 1999 Am J. Physiol. 276:G58-G63). Four experimental groups were: control (buffer alone), cholera-treated, cholera-treated+inhibitor (150 μg/kg intraperitoneal 2 min before gavage), and inhibitor alone. After six hours mice were euthanized and the small intestine (from pylorus to cecum) was exteriorized and stripped of associated mesenteric and connective tissues. The intestine was weighed, then opened longitudinally to remove lumenal fluid (by blotting), and weighed again. Fluid accumulation was computed from the ratio in intestinal weight before and after lumenal fluid removal. For measurement of short-circuit current, strips of rat colon were isolated, stripped of muscle layers by blunt dissection, mounted in Ussing chambers (area 0.7 cm²), and bathed in oxygenated bicarbonate Ringers solution containing 10 μM indomethacin. Short-circuit current was measured after inhibition of Na⁺ current by amiloride (10 μM), followed by stimulation by forskolin (20 μM) and subsequent inhibitor addition.

### Example 1

Screening of CFTR Inhibitors

The primary screening technique used to identify the compounds of the invention was designed to identify inhibitors of CFTR Cl⁻ conductance by direct CFTR-inhibitor interaction. CFTR was pre-stimulated in CFTR-expressing FRT cells by an activating cocktail containing forskolin, IBMX and apigenin, as shown schematically in FIG. 1A. The activation of CFTR by multiple mechanisms (cAMP elevation, phosphodiesterase inhibition, and direct CFTR binding) allowed identification of inhibitors that blocked the CFTR Cl⁻ transporting pathway directly rather than more proximal step(s) in a signaling pathway. The FRT cells co-expressed a yellow fluorescent protein-based Cl⁻/I⁻ sensor that provided a quantitative fluorescence read-out of inhibition potency (See, e.g., Jayaraman et al., 2000, J. Biol. Chem. 275:6047-6050; Galietta et al., 2001, Am. J. Physiol. 281:C1734-C1742.). After CFTR pre-stimulation and compound addition, cells were subjected to an inwardly-directed I⁻ gradient to drive I⁻ influx and produce decreasing fluorescence. Each assay consisted of recording baseline fluorescence for 2 seconds, followed by 12 seconds of continuous recording of fluorescence after rapid addition of the I⁻ containing solution. Compounds were tested separately at 10 μM concentration in a 96-well format utilizing a fully-automated high-throughput screening apparatus (see Example 2 below).

FIG. 1B graphically illustrates representative curves, as relative YFP fluorescence versus time, from the primary screen of 50,000 compounds using the assay of FIG. 1A. As quantified from the slope of the decreasing fluorescence after I⁻ addition, 49,993 compounds had no significant effect on the kinetics of I⁻ influx (<10% decrease in slope). Seven compounds produced a small decrease in negative slope (10-52%), nearly all of which had a similar core structure consisting of a 2-thioxo-4-thiazolidinone heterocycle with substituted phenylmethylene and phenyl moieties (FIG. 1C). More than 250 analogs having thiazolidinone core structure were subsequently screened to identify the most potent CFTR inhibitors.

FIG. 1D shows the most effective thiazolidinone CFTR inhibitors identified in the screening were 3-[(3-trifluoromethyl)phenyl]-5-[(4-carboxyphenyl)methylene]-2-thioxo-4-thiazolidinone (referred to herein as CFTRᵢₙₕ-172), along with five analogs having significant inhibitory potencies. Thus the following compounds were identified as CFTR inhibitors: 3-[( 3-trifluoromethyl)phenyl]-5-[(4-carboxyphenyl)methylene]-2-thioxo-4-thiazolidinone (CFTRᵢₙₕ-172); 3-[(3-trifluoromethyl)phenyl]-5-[(4-nitrophenyl)methylene]-2-thioxo-4-thiazolidinone(CFTRᵢₙₕ-020); 3-[(3-trifluoromethyl)phenyl]-5-[(4-oxycarboxyphenyl)methylene]-2-thioxo-4-thiazolidinone (CFTRᵢₙₕ-029); 3-[(3-trifluoromethyl)phenyl]-5-[(3,4-dihydroxyphenyl)methylene]-2-thioxo-4-thiazolidinone (CFTRᵢₙₕ-185), 3-[(3-trifluoromethyl)phenyl]-5-[(3,5-dibromo-4-hydroxyphenyl)methylene]-2-thioxo-4-thiazolidinone (CFTRᵢₙₕ-214) and 3-[(3-trifluoromethyl)phenyl]-5-[(3-bromo-4-hydroxy-5-nitrophenyl)methylene]-2-thioxo-4-thiazolidinone (CFTRᵢₙₕ-236). The most effective CFTR inhibitors included one or more electronegative groups such as a 3-trifluoromethyl group on ring 1, and electronegative or polar substituents on ring 2 as discussed above. CFTRᵢₙₕ-172 was selected for further analysis. The relative potencies were: 0.2 (CFTRᵢₙₕ-020), 0.3 (CFTRᵢₙₕ-029), 1.0 (CFTRᵢₙₕ-172), 0.2 (CFTRᵢₙₕ-185), 0.1 (CFTRᵢₙₕ-214), and 0.1 (CFTRᵢₙₕ-236).

To examine the effect of ring position of the trifluoromethyl and carboxyl substituents, 8 analogs of CFTRᵢₙₕ-172 were synthesized in which the substituents were moved to each unique position on rings 1 (trifluoromethyl) and 2 (carboxy). Compared to CFTRᵢₙₕ-172 (potency 1.0), the relative inhibitory potencies of the 3-[(a-trifluoromethyl)phenyl]-5-[(b-carboxyphenyl) methylene]-2-thioxo-4-thiazolidinone analogs were: 0.69 (a=2, b=2), 0.70 (2, 3), 0.66 (2, 4), 0.74 (3, 2), 0.90 (3, 3), 0.67 (4, 2), 0.64 (4, 3) and 0.56 (4, 4).

### Example 2

Characterization of CFTRᵢₙₕ-172

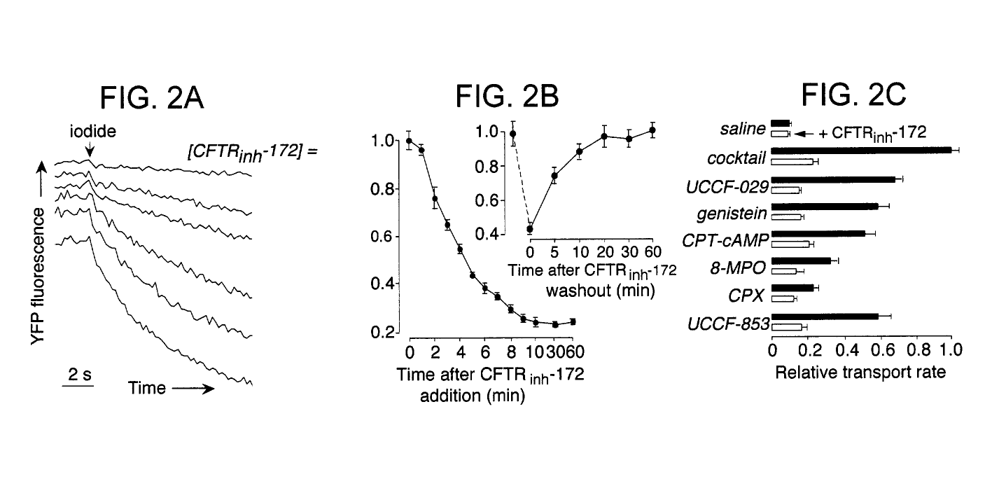

The level of CFTR inhibition for specific dosages of the subject thiazolidinone compounds was determined using the fluorescence assay shown in FIG. 1A and described above. FIG. 2A shows dose-inhibition data for CFTRᵢₙₕ-172 as relative YFP fluorescence versus time. Significant CFTR inhibition was seen at 0.3-0.6 μM concentrations of this thiazolidinone compound. FIG. 2B shows that inhibition by CFTRᵢₙₕ-172 (shown graphically as relative transport rate versus time after addition or washout) was complete in ˜10 min (t_{1/2} 4 min) and was reversed after washout with t_{1/2} ˜5 min (inset). The relative transport rates illustrated in FIG. 2C show that CFTRᵢₙₕ-172 effectively inhibited CFTR activation by multiple types of agonists that were not included in the activating cocktail used for initial screening. These agonists included genistein, CPT-cAMP, CPX, 8-MPO and the potent benzoflavone CFTR activator UCCF-029 (2-(4-pyridinium)benzo[h]4H-chromen-4-one bisulfate) and the benzimidazolone CFTR activator UCCF-853 (see Galietta, et al., 2001, J. Biol. Chem. 276:19723-19728).

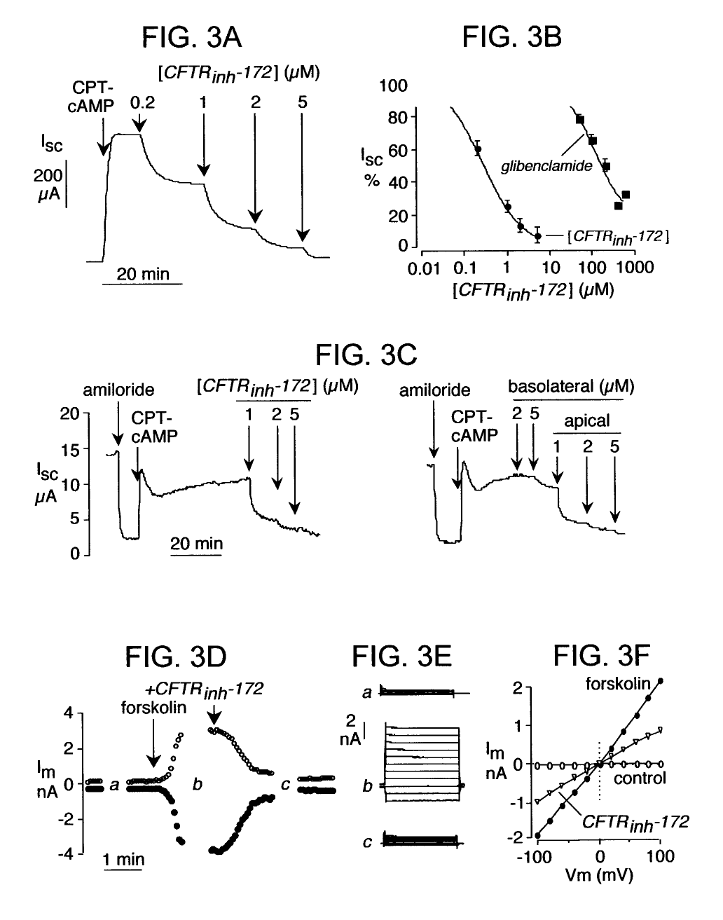

Electrophysiology experiments were also carried out to establish the inhibitory potency and specificity of CFTRᵢₙₕ-172. FIG. 3A shows the rapid, dose-dependent inhibition of short-circuit current in CFTR-expressing FRT cells with CFTRᵢₙₕ-172 added to the solution bathing the apical cell surface. FIG. 3B shows the average dose-inhibition relationships of CFTRᵢₙₕ-172 (K_{d}˜300 nM, Hill coefficient ˜1) and glibenclamide (K_{d}˜200 μM) tested under identical conditions.

Similar inhibitory potencies for this thiazolidinone were found in cells that natively express wildtype CFTR, including T84 cells and primary cultures of human bronchial epithelial cells, as well as in transfected FRT cells expressing G551D-CFTR and ΔF508-CFTR (after low temperature correction). For studies in bronchial cells, the Na⁺ channel was blocked with amiloride so that baseline current is largely CFTR-dependent. After maximal CFTR activation by a CPT-cAMP, application of CFTRᵢₙₕ-172 from the apical side inhibited short-circuit current strongly (FIG. 3C, left). CFTRᵢₙₕ-172 also inhibited short-circuit current when added from the basolateral side (FIG. 3C, right).

Whole-cell membrane currents were measured in CFTR-expressing FRT cells as shown in FIG. 3D. Stimulation by 5 μM forskolin produced a membrane current of 381±47 pA/pF (n=4) at +100 mV (total membrane capacitance 21±3 μF). The current-voltage relationship was linear as expected for a pure CFTR current (FIG. 3F). Extracellular perfusion with 2 μM CFTRᵢₙₕ-172 produced a rapid reduction in current at all membrane potentials, suggesting voltage-independent CFTR inhibition. The lack of voltage-dependence of channel block was confirmed using a lower concentration of CFTRᵢₙₕ-172 (0.2 μM) to obtain ˜50 % inhibition (FIG. 3F).

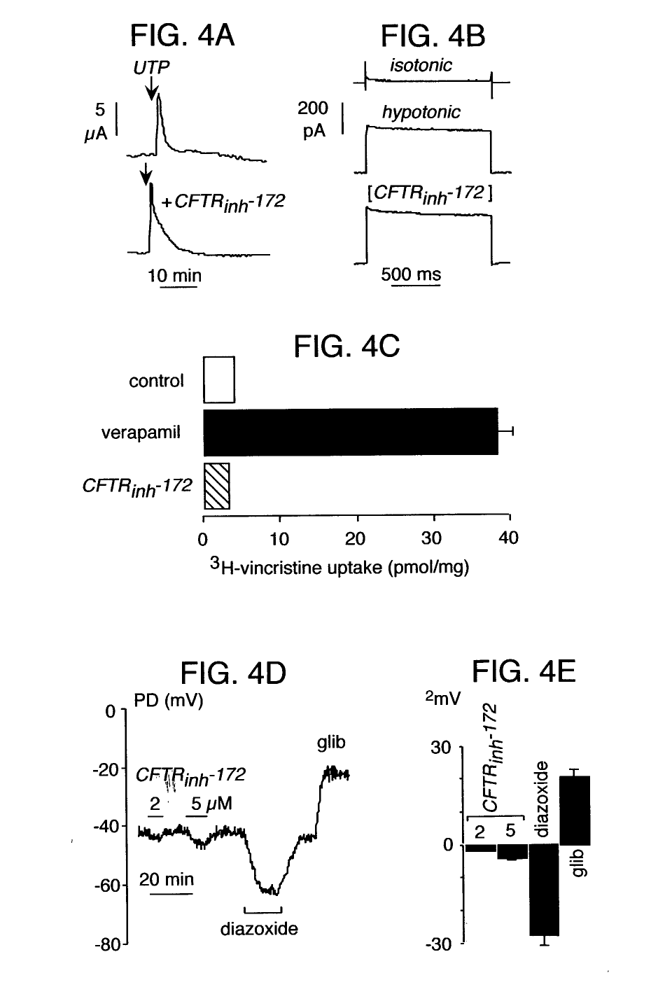

The specificity of CFTRᵢₙₕ-172 for inhibition of CFTR was also examined. Two non-CFTR Cl⁻ channes were studied. CFTRᵢₙₕ-172 at 5 μM concentration did not inhibit Ca²⁺ activated Cl⁻ secretion produced by addition of UTP (100 μM) to the apical bathing solution in polarized human bronchial epithelial cells (FIG. 4A). Maximal UTP-dependent short-circuit currents were 9.9±0.5 μA/cm² and 10.0±0.2 μA/cm² in the absence and presence of CFTRᵢₙₕ-172, respectively (SE, n=4). CFTRᵢₙₕ-172 at 5 μM also did not block volume-activated Cl⁻ currents elicited in FRT cells by extracellular perfusion with a 250 mosM/kg hypotonic solution (FIG. 4B).

The activity of a CFTR homolog, the ATP-binding cassette transporter MDR-1 (multi-drug resistance protein-1), was measured in 9HTEo-/Dx which overexpress MDR-1 (Rasola et al. 1994 J. Biol. Chem. 269:1432-1436). Vincristine accumulation, which is inversely related to active drug extrusion by MDR-1, was strongly increased by the MDR-1 inhibitor verapamil (100 μM) (FIG. 4C). CFTRᵢₙₕ-172 (5 μM) did not affect vincristine accumulation and thus did not inhibit MDR-1.

Another homolog of CFTR is the sulphonylurea receptor (SUR) which regulates the activity of ATP-sensitive K⁺ channels (K-ATP channel) (Aguilar-Bryan and Bryan 1999 Endocr. Rev. 20:101-135). SUR1 is expressed in pancreatic β-cells where it controls membrane potential and insulin release. Sulphonylureas, like glibenclamide, cause insulin release (and a hypoglycemic response) by blocking K-ATP channels and membrane depolarization. To determine whether CFTRᵢₙₕ-172 also blocks K-ATP channels, membrane potential in a rat pancreatic β cell line, INS-1, was measured (FIG. 4D, FIG. 4E). In contrast to large membrane depolarization caused by glibenclamide, CFTRᵢₙₕ-172 (2 and 5 μM) did not depolarize membrane potential. CFTRᵢₙₕ-172 at 5 μM caused a small hyperpolarization that was much less than that caused by the K-ATP channel activator diazoxide (100 μM). Additional studies indicated that CFTRᵢₙₕ-172 at 5 μM did not block a water channel (AQP1), urea transporter (UT-B), Na⁺/H⁺ exchanger (NEE3) and Cl⁻/HCO₃⁻ exchanger (AEI).

Further analysis showed that 5 μM CFTRᵢₙₕ-172 did not affect cellular cAMP production or phosphatase activity. In FRT cells, basal cAMP content was 225±22 fmol/well, which increased at 30 min after stimulation by 20 μM forskolin to 1290±190 fmol/well (no inhibitor) and 1140±50 (+CFTRᵢₙₕ-172) (n=3). As judged using the dihydrorhodamine assay, CFTRᵢₙₕ-172 was non-toxic to FRT cells after 24 hours at concentrations up to 100 μM. In mice, intraperitoneal injection of 1000 μg/kg CFTRᵢₙₕ-172 daily for 7 days did not cause overt toxicity. Food and water intake were not diminished, and serum electrolyte concentrations, glucose, liver function indices, serum creatinine, amylase and hematocrit were not changed. In addition, a single very large systemic dose of CFTRᵢₙₕ-172 (10 mg/kg) did not cause overt toxicity.

### Example 3

In Vivo Efficacy

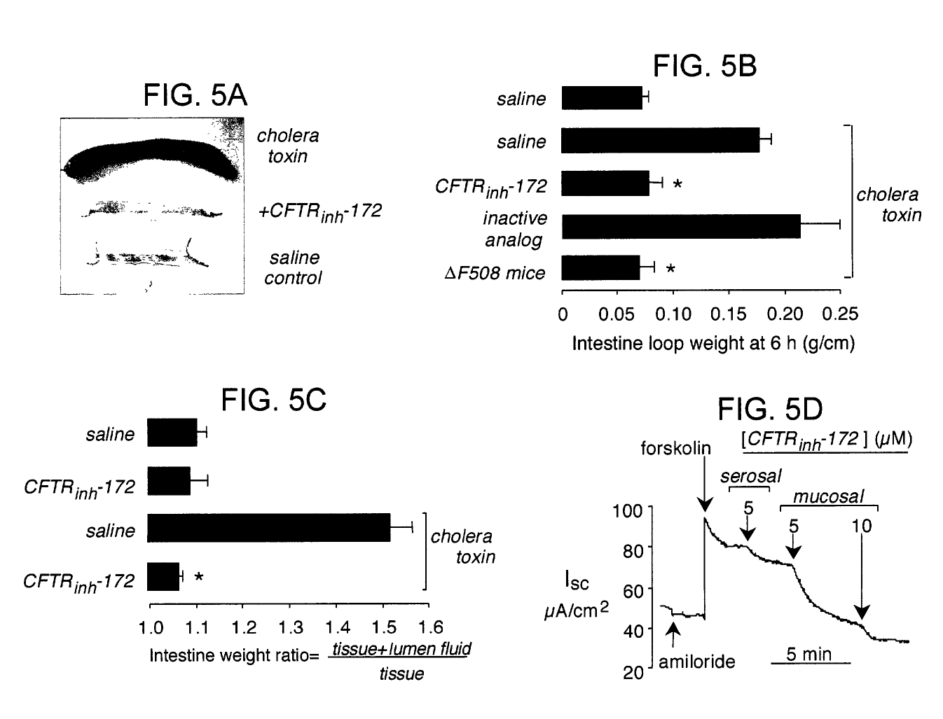

The efficacy of CFTRᵢₙₕ-172 was tested in vivo using two assays of cholera toxin-induced intestinal fluid secretion, and in isolated intestine by short-circuit analysis. In the first assay, a series of closed loops of small intestine were created in vivo and the lumens of alternate loops were injected with small volumes of saline or saline containing cholera toxin. Luminal fluid accumulation was determined after 6 hours. As seen visually in FIG. 5A, there was marked fluid accumulation and distention in cholera toxin-treated loops, whereas adjacent control (saline) loops remained empty. A single administration of CFTRᵢₙₕ-172 (150 μg/kg intraperitoneal) prior to cholera toxin infusion effectively prevented fluid accumulation in the toxin-treated intestinal loops.

Data from a series of these experiments is summarized graphically in FIG. 5B. CFTRᵢₙₕ-172 significantly reduced fluid secretion to that in saline control loops where an inactive thiazolidinone analog did not inhibit fluid secretion. As suggested from previous data (Gabriel et al. 1994 Science 266:107-109), cholera toxin-treated loops of intestine from homozygous ΔF508-CFTR mice also remained empty, indicating the involvement of CFTR in intestinal fluid secretion. In the second assay, intestinal fluid secretion was induced by oral administration of cholera toxin (10 μg) and CFTRᵢₙₕ-172 was administered systemically. After six hours there was marked accumulation of fluid as measured by weighing the entire small intestine. CFTRᵢₙₕ-172 administration remarkably reduced intestinal fluid accumulation as seen visually and quantified by the ratio of intestinal weight before vs. after luminal fluid removal (FIG. 5C).

FIG. 5D shows CFTRᵢₙₕ-172 inhibition of short-circuit current across intact rat colonic mucosa. After inhibition of Na⁺ current by amiloride, forskolin produced a prompt increase in short-circuit current. CFTRᵢₙₕ-172 added to the mucosal solution inhibited short-circuit current with greater efficacy than when added to the serosal solution, which may be related to impaired access to colonic epithelial cells through the residual submucosal tissue. Addition of 5 μM CFTRᵢₙₕ-172 to the mucosal solution alone reduced short-circuit current by >80%. These results provide electrophysiological evidence for CFTR Cl⁻ channel inhibition by CFTRᵢₙₕ-172 in intestine.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a subject having a condition associated with aberrant ion transport by cystic fibrosis transmembrane conductance regulator (CFTR) in a subject, the method comprising:
administering to the subject an efficacious amount of a thiazolidinone compound;
wherein CFTR ion transport is inhibited and the condition is treated.

2. The method of claim 1, wherein the aberrantly increased CFTR ion transport is associated with diarrhea.

3. The, method of claim 2, wherein the diarrhea is secretory diarrhea.

4. The method of claim 1, wherein said thiazolidinone compound comprises a 3-aryl-5-arylmethylene-2-thioxo-4-thiazolidinone.

5. The method of claim 1, wherein said thiazolidinone compound is selected from the group consisting of: 3-[(3-trifluoromethyl)phenyl]-5-[(4-nitrophenyl)methylene]-2-thioxo-4-thiazolidinone; 3-[(3-trifluoromethyl)phenyl]-5-[(4-oxycarboxyphenyl)methylene]-2-thioxo-4-thiazolidinone; 3-[(3-trifluoromethyl)phenyl]-5-[(4-carboxyphenyl)methylene]-2-thioxo-4-thiazolidinone; 3-[(3-trifluoromethyl)phenyl]-5-[(3,4-dihydroxyphenyl)methylene]-2-thioxo-4-thiazolidinone; 3-[(3-trifluoromethyl)phenyl]-5-[(3,5-dibromo-4-hydroxyphenyl)methylene]-2-thioxo-4-thiazolidinone; and 3-[(3-trifluoromethyl)phenyl]-5-[(3-bromo-4-hydroxy-5-nitrophenyl)methylene]-2-thioxo-4-thiazolidinone.

6. The method of claim 1, wherein said thiazolidinone compound comprises the formula: 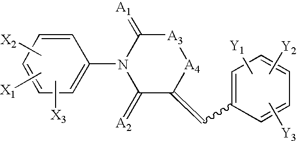
wherein X₁, X₂ and X₃ each individually are hydrogen, any organic group, any halo group, a nitro group, an azo group, a hydroxyl group or a thio group, Y₁, Y₂ and Y₃ each individually are hydrogen, any organic group, any halo group, a nitro group, an azo group, a hydroxyl group or a thio group, A₁ and A₂ each individually are oxygen or sulfur, A₃ is sulfur or selenium, and A₄ comprises one or more carbons or heteroatoms and may be present or absent.

7. The method of claim 1, wherein said thiazolidinone compound comprises the formula: 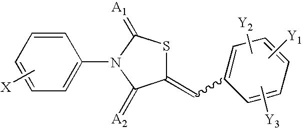
wherein X is hydrogen, any organic group, any halo group, a nitro group, an azo group, a hydroxyl group or a thio group, Y₁, Y₂ and Y₃ each individually are hydrogen, any organic group, any halo group, a nitro group, an azo group, a hydroxyl group or a thio group, and A₁ and A₂ each independently are oxygen or sulfur.

8. The method of claim 7, wherein X is an electronegative group.

9. The method of claim 8, wherein X is selected from the group consisting of a perfluoroalkyl group and a fluoro group.

10. The method of claim 9, wherein Y1 is selected from the group consisting of alkyl, hydroxyl, carboxyl, nitro, carbonate, carbamate, alkoxy, alkylcarbonyl, and halo groups.

11. The method of claim 8, wherein X is a 3-trifluoromethyl group.

12. The method of claim 7, wherein Y1 is a hydroxyl group.

13. The method of claim 12, wherein Y2 is a hydroxyl group.

14. The method of claim 12, wherein Y2 is a bromo group.

15. The method of claim 12, wherein Y3 is a nitro group.

16. The method of claim 1, wherein said thiazolidinone compound comprises the formula: 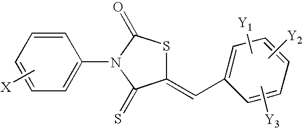
wherein X is hydrogen or any organic group, Y1, Y2 and Y3 each individually are hydrogen or any organic group.

17. The method of claim 1, wherein said thiazolidinone compound comprises the formula: 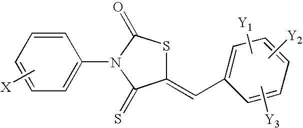
wherein X is any electronegative group or electron withdrawing group, and Y₁, Y₂ and Y₃ each individually are a hydrogen, alkyl, hydroxyl, carboxyl, nitro, carbonate, carbamate, alkoxy, alkylcarbonyl, or halo group.

18. The method of claim 17, wherein X is a trifluoromethyl group.

19. The method of claim 18, wherein X is a 3-trifluoromethyl group.

20. The method of claim 1, wherein said thiazolidinone compound comprises a formula selected from the group consisting of:

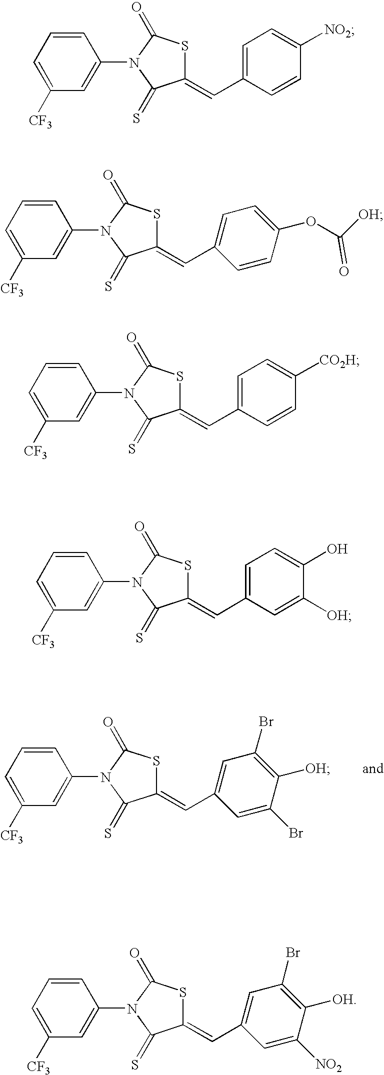

21. A method for inhibiting cystic fibrosis transmembrane conductance regulator protein in cells of a subject, comprising contacting said cells with an efficacious amount of a thiazolidinone compound.

22. The method of claim 21, wherein said thiazolidinone compound comprises a 3-aryl-5-arylmethylene-2-thioxo-4-thiazolidinone.

23. The method of claim 21, wherein said thiazolidinone compound is selected from the group consisting of: 3-[(3-trifluoromethyl)phenyl]-5-[(4-nitrophenyl)methylene]-2-thioxo-4-thiazolidinone; 3-[(3-trifluoromethyl)phenyl]-5-[(4-oxycarboxyphenyl)methylene]-2-thioxo-4-thiazolidinone; 3-[(3-trifluoromethyl)phenyl]-5-[(4-carboxyphenyl)methylene]-2-thioxo-4-thiazolidinone; 3-[(3-trifluoromethyl)phenyl]-5-[(3,4-dihydroxyphenyl)methylene]-2-thioxo-4-thiazolidinone; 3-[(3-trifluoromethyl)phenyl]-5-[(3,5-dibromo-4-hydroxyphenyl)methylene]-2-thioxo-4-thiazolidinone; and 3-[(3-trifluoromethyl)phenyl]-5-[(3-bromo-4-hydroxy-5-nitrophenyl)methylene]-2-thioxo-4-thiazolidinone.

24. The method of claim 21, wherein said thiazolidinone compound comprises the formula: 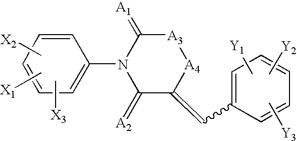
wherein X₁, X₂ and X₃ each individually are hydrogen, any organic group, any halo group, a nitro group, an azo group, a hydroxyl group or a thio group, Y₁, Y₂ and Y₃ each individually are hydrogen, any organic group, any halo group, a nitro group, an azo group, a hydroxyl group or a thio group, A₁ and A₂ each individually are oxygen or sulfur, A₃ is sulfur or selenium, and A₄ comprises one or more carbons or heteroatoms and may be present or absent.

25. The method of claim 21, wherein said thiazolidinone compound comprises the formula: 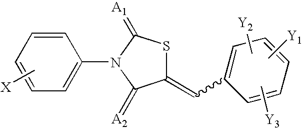
wherein X is hydrogen, any organic group, any halo group, a nitro group, an azo group, a hydroxyl group or a thio group, Y₁, Y₂ and Y₃ each individually are hydrogen, any organic group, any halo group, a nitro group, an azo group, a hydroxyl group or a thio group, and A₁ and A₂ each independently are oxygen or sulfur.

26. The method of claim 25, wherein X is an electronegative group.

27. The method of claim 26, wherein X is selected from the group consisting of a perfluoroalkyl group and a fluoro group.

28. The method of claim 25, wherein Y1 is selected from the group consisting of alkyl, hydroxyl, carboxyl, nitro, carbonate, carbamate, alkoxy, alkylcarbonyl, and halo groups.

29. The method of claim 26, wherein X is a 3-trifluoromethyl group.

30. The method of claim 25, wherein Y1 is a hydroxyl group.

31. The method of claim 30, wherein Y2 is a hydroxyl group.

32. The method of claim 30 wherein Y2 is a bromo group.

33. The method of claim 30, wherein Y3 is a nitro group.

34. The method of claim 21, wherein said thiazolidinone compound comprises the formula: 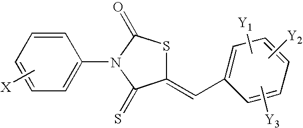
wherein X is hydrogen or any organic group, Y1, Y2 and Y3 each individually are hydrogen or any organic group.

35. The method of claim 21, wherein said thiazolidinone compound comprises the formula: 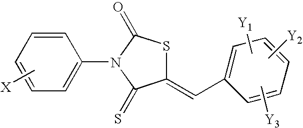
wherein X is any electronegative group or electron withdrawing group, and Y₁, Y₂ and Y₃ each individually are a hydrogen, alkyl, hydroxyl, carboxyl, nitro, carbonate, carbamate, alkoxy, alkylcarbonyl, or halo group.

36. The method of claim 35, wherein X is a trifluoromethyl group.

37. The method of claim 36, wherein X is a 3-trifluoromethyl group.

38. The method of claim 21, wherein said thiazolidinone compound comprises a formula selected from the group consisting of:

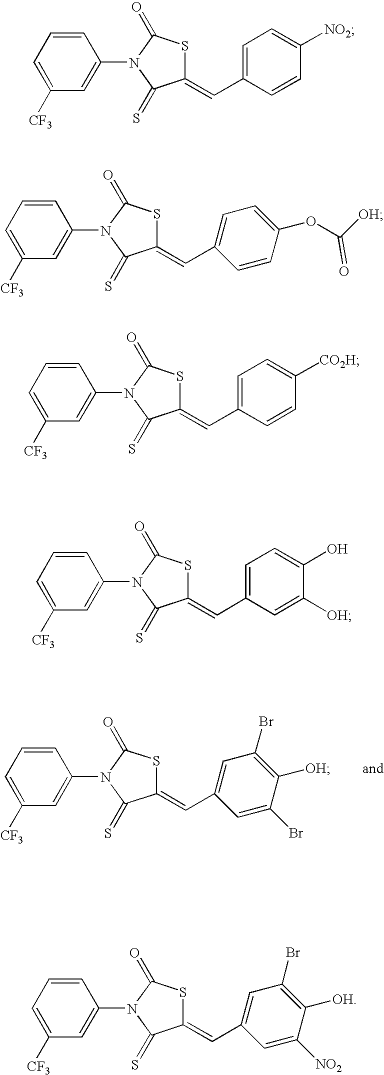

39. The method of claim 21, wherein said contacting said cells is carried out in vivo in a subject.

40. The method of claim 21, wherein said contacting said cells comprises ingesting, by said subject, said thiazolidinone compound.

41. The method of claim 40, wherein said ingesting further comprises ingesting a pharmaceutically acceptable carrier together with said thiazolidinone compound.

42. A pharmaceutical composition comprising a thiazolidinone compound together with at least one of a pharmaceutically acceptable carrier, a pharmaceutically acceptable diluent, a pharmaceutically acceptable excipient and a pharmaceutically acceptable adjuvant, said thiazolidinone compound selected from the group consisting of: 3-[(3-trifluoromethyl)phenyl]-5-[(4-nitrophenyl)methylene]-2-thioxo-4-thiazolidinone; 3-[(3-trifluoromethyl)phenyl]-5-[(oxycarboxyphenyl)methylene]-2-thioxo-4-thiazolidinone; 3-[(3-trifluoromethyl)phenyl]-5-[(4-carboxyphenyl)methylene]-2-thioxo-4-thiazolidinone; 3-[(3-trifluoromethyl)phenyl]-5-[(3,4-dihydroxyphenyl)methylene]-2-thioxo-4-thiazolidinone; 3-[(3-trifluoromethyl)phenyl]-5-[(3,5-dibromo-4-hydroxyphenyl)methylene]-2-thioxo-4-thiazolidinone; and 3-[(3-trifluoromethyl)phenyl]-5-[(3-bromo-4-hydroxy-5-nitrophenyl)methylene]-2-thioxo-4-thiazolidinone.

43. The composition of claim 42, wherein said composition does not contain dimethyl sulfoxide.

44. A pharmaceutical composition comprising a thiazolidinone compound together with at least one of a pharmaceutically acceptable carrier, a pharmaceutically acceptable diluent, a pharmaceutically acceptable excipient and a pharmaceutically acceptable adjuvant, said thiazolidinone compound having a formula: 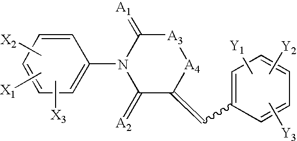
wherein X₁, X₂ and X₃ each individually are hydrogen, any organic group, any halo group, a nitro group, an azo group, a hydroxyl group or a thio group, Y₁, Y₂ and Y₃ each individually are hydrogen, any organic group, any halo group, a nitro group, an azo group, a hydroxyl group or a thio group, A₁ and A₂ each individually are oxygen or sulfur, A₃ is sulfur or selenium, and A₄ comprises one or more carbons or heteroatoms and may be present or absent.

45. The composition of claim 44, wherein said thiazolidinone compound comprises the formula: 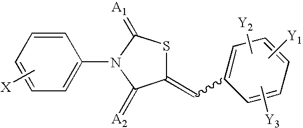
wherein X is hydrogen, any organic group, any halo group, a nitro group, an azo group, a hydroxyl group or a thio group, Y₁, Y₂ and Y₃ each individually are hydrogen, any organic group, any halo group, a nitro group, an azo group, a hydroxyl group or a thio group, and A₁ and A₂ each independently are oxygen or sulfur.

46. The composition of claim 45, wherein X is an electronegative group.

47. The composition of claim 46, wherein X is selected from the group consisting of a perfluoroalkyl group and a fluoro group.

48. The composition of claim 45, wherein Y1 is selected from the group consisting of alkyl, hydroxyl, carboxyl, nitro, carbonate, carbamate, alkoxy, alkylcarbonyl, and halo groups.

49. The composition of claim 45, wherein X is a 3-trifluoromethyl group.

50. The composition of claim 45, wherein Y1 is a hydroxyl group.

51. The composition of claim 50, wherein Y2 is a hydroxyl group.

52. The composition of claim 50, wherein Y2 is a bromo group.

53. The method of claim 52, wherein Y3 is a nitro group.

54. The composition of claim 44, wherein said thiazolidinone compound comprises the formula: 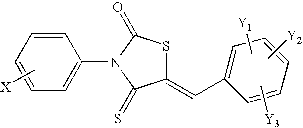
wherein X is hydrogen or any organic group; and Y1, Y2, and Y3 each individually are hydrogen or any organic group.

55. The composition of claim 54, wherein said thiazolidinone compound comprises the formula: 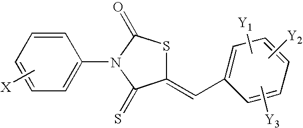
wherein X is any electronegative group or electron withdrawing group, and Y₁, Y₂ and Y₃ each individually are a hydrogen, alkyl, hydroxyl, carboxyl, nitro, carbonate, carbamate, alkoxy, alkylcarbonyl, or halo group.

56. The composition of claim 55, wherein X is a trifluoromethyl group.

57. The composition of claim 55, wherein X is a 3-trifluoromethyl group.

58. The composition of claim 44, wherein said composition does not contain dimethyl sulfoxide.

59. A non-human animal having a cystic fibrosis transmembrane conductance regulator (CFTR) deficiency, wherein the deficiency is produced by administration of a thiazolidinone compound to the animal in an amount effective to inhibit CFTR ion transport.

60. The non-human animal of claim 59, wherein the animal is a mammal.

61. The non-human animal of claim 60, wherein the mammal is a non-human primate, rodent, ungulate, or avian.

62. The non-human animal of claim 59, wherein the animal has a phenotype similar to cystic fibrosis.
